(19) United States
(12) Patent Application Publication
(10) Pub. No.: US 2018/0116986 A1
(43) Pub. Date: May 3, 2018

(54) COMPOSITION OF NOVEL POWDER FORMULATIONS OF TRANEXAMIC ACID

(71) Applicants: HEMANT N. JOSHI, PARSIPPANY, NJ (US); HARSHADA N. SANT, HARRISON, NJ (US); AMITKUMAR LAD, NEW MILFORD, NJ (US)

(72) Inventors: HEMANT N. JOSHI, PARSIPPANY, NJ (US); HARSHADA N. SANT, HARRISON, NJ (US); AMITKUMAR LAD, NEW MILFORD, NJ (US)

(21) Appl. No.: 15/337,904

(22) Filed: Oct. 28, 2016

(51) Int. Cl. A61K 31/195; A61K 47/36; A61K 31/7036; A61K 38/12; A61K 31/18; A61K 31/167; A61K 38/57; A61K 38/14; A61K 36/9066; A61K 31/4709; A61K 47/40; A61K 38/48; A61K 31/197; A61K 31/5375; A61K 47/38; A61K 9/00; A61K 9/14

(52) U.S. Cl. CPC A61K 31/195; A61K 9/0014; A61K 31/7036; A61K 38/12; A61K 31/18; A61K 31/167; A61K 38/57; A61K 38/14; A61K 36/9066; A61K 31/4709; A61K 47/40; A61K 38/4873; A61K 31/197; A61K 31/5375; A61K 47/38; A61K 9/0073; A61K 9/0043; A61K 9/14; C12Y 304/22004; A61K 47/36

(57) ABSTRACT

Powder composition of Tranexamic acid have been provided for the treatment of wound and bleeding. The powder composition may also contain aprotinin and epsilon-aminocaproic acid as active antifibrinolytic agent. The composition may also contain antibiotic(s), anti-inflammatory agent(s), local anesthetic(s) and hydrophilic polymer(s). The powder composition in this patent application is applied to mucosal or non-mucosal surfaces, but it is not for an oral administration.

### FIELD OF INVENTION

The present invention relates to powder compositions of Tranexamic Acid (TXA) as a novel delivery system and methods of treating bleeding and wounds.

### BACKGROUND OF INVENTION

Tranexamic Acid (TXA) is a synthetic derivative of the amino acid lysine with antifibrinolytic activity. With a strong affinity for the five lysine-binding sites of plasminogen, TXA competitively inhibits the activation of plasminogen to plasmin, resulting in inhibition of fibrinolysis; at higher concentrations, this agent noncompetitively inhibits plasmin. This agent has a longer half-life, is approximately ten times more potent, and is less toxic than aminocaproic acid, which possesses similar mechanisms of action.

TXA is used to control excess bleeding, for example, excess bleeding that occurs during dental procedures in hemophiliacs, heavy bleeding during menstruation (menorrhagia), during surgery, trauma, etc., Many TXA formulations are available in the market and are tablets (Lysteda) and injectables (Cyklokapron). Currently, T×A tablets, 650 mg, are supplied by Ferring Pharms, Actavis Labs, Apotex and Mylan labs. TXA injections (100 mg/mL) are supplied by Pharmacia and Upjohn, Acic Fine Chem, Akorn, Aurobindo Pharma, Empure Pharma, Fresenius Kabi, Luitpold Pharma, Mylan, North Creek Pharma, Versapharm, and X-Gen Pharma. Wound is an injury, usually involving division of tissue or rupture of the integument or mucous membrane, due to external violence or some mechanical agency rather than disease. Typically, wound is related to one in which the skin is cut or broken. For this patent, wounds are considered as any kinds of wounds—on the skin or internal.

The result of a wound is mild to severe blood loss and a higher chance of getting microbial infections. Several wound healing treatments are already available. TXA is a drug, which helps the wound healing process.

U.S. Pat. No. 9,301,936 presented pharmaceutical formulations comprising tranexamic acid, kits thereof, and methods for treating bleeding by local administration. If the compositions of the disclosure are to be administered topically, the compositions can be formulated in the form of an ointment, cream, patch such as transdermal patch, lotion, gel, shampoo, spray, aerosol, solution, sponge, film, plaster, surgical dressing, bandage, emulsion, or other form well-known to one of skill in the art. For non-sprayable topical dosage forms, viscous to semi-solid or solid forms comprising a carrier or one or more excipients compatible with topical application and having a dynamic viscosity preferably greater than water are typically employed. Suitable formulations include, paste, patch, lotion, gel, solution, suspension, cream, ointment, liniments, shampoo, hydrogel, liposomes, spray, aerosol, sponge, film, plaster, a surgical dressing, bandage, or an emulsion, which are, if desired, sterilized or mixed with auxiliary agents (e.g., preservatives, stabilizers, wetting agents, buffers, or salts) for influencing various properties, such as, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as FREON®) or in a squeeze bottle. Moisturizers or humectants can also be added to pharmaceutical compositions and dosage forms, if desired. The main claim included 1-70% TXA, one or more antibiotic, optionally one or more anesthetic, optionally one or more non-steroid anti-inflammatory drug and suitable excipients.

US 20140220136 A1 describes the system and method of artificially retarding fibrin-based blood clot degradation via the sustained release of a protease inhibitor, such as, for example, aprotinin or TXA. The formulation along with fibrinogen and thrombin is applied to a wound site where an outer surface of the polymer microsphere degrades in a proteolytic environment to expose and release the incorporated protease inhibitor to the surrounding hydrogel or sealant or clot matrix at the wound site.

US 20140219939 A1 provides an external-use liquid skin-conditioning composition that not only reduces the sticky and friction sensations caused by the inclusion of TXA, but also has a rich body without feeling slimy and softens the skin after use. The composition comprises (A) 0.5 to 5 mass % of at least one selected from a group consisting of TXA and derivatives thereof and (B) 0.005 to 1.5 mass % of carboxymethylcellulose.

US 20140039054 A1 described oral combination drug formulation comprising a non-steroidal anti-inflammatory drug and a complementary low dose of TXA for the treatment of menstrual pain accompanied with excessive menstrual blood loss.

US 20130018020 A1 describes a formulation containing melanin production inhibitor. It contains TXA and nicotinic acid amide as active ingredients, which inhibit the formation of skin melanocytes. It has the effects of reducing liver spots, blemishes, freckles and post-inflammatory hyperpigmentation, improving skin tone and texture and whitening the skin.

Certain key performance characteristics are required for wound healing products: they must absorb and retain exudate, keep harmful chronic wound exudate away from the surrounding skin, perform efficiently when used under compression, be easy to remove and be demonstrated as cost-effective. Wound dressings exhibit various fluid-handling mechanisms: absorption, gelling, retention and moisture vapor transmission.

TXA was used to reduce bleeding in burns surgery (J. Plast. Reconstr. Aesthet.Surg. 65: 684-686, 2012). TXA has been used in an ad hoc manner during plastic surgery where there is a risk of blood loss. It was reported that the topical use of TXA was preferred over the systemic administration. Not sufficient literature is available to ascertain the safety of TXA usage to treat burns.

Based on results of CRASH-2 trial, TXA has been recommended to the WHO's list as an essential medicine (J. Trauma Acute Care Surg. 74: 1575-1586, 2013).

TXA has been reported to be safer with topical administration than with intravenous administration in total knee arthroplasty. However, the most effective administration route of TXA in total hip arthroplasty remains controversial. The study compared the effectiveness of topical TXA administration with that of intravenous TXA administration in total hip arthroplasty (J. of Orthopaedic Science, 21: 44-47, 2016).

Jean Wong and their group have reported the efficacy and safety of the topical application of TXA on postoperative blood loss in patients undergoing primary unilateral total knee arthroplasty with cement (J Bone Joint Surg Am, 92 (15): 2503-2513, 2010).

As mentioned earlier, only tablets and injectable products of TXA have been approved in the US. In medical cosmetics, small amount of TXA can bleach pigmented spots and reduce redness. Imprimis Pharmaceuticals has obtained a patent for local application of TXA (U.S. Pat. No. 9,301,936), but they have not filed and launched the product. The inventors have claimed ointment, cream, liniment, paste, patch, lotion, gel, shampoo, hydrogel, liposome, spray, aerosol, solution, sponge, film, plaster, surgical dressing, bandge or an emulsion. No powder formulation of TXA has been available in the US, European and Indian markets.

### SUMMARY OF INVENTION

The main objective of this patent application is to provide powder compositions consisting essentially of a therapeutically effective amount of TXA and suitable excipients that facilitate local administration and methods of use to treat wounds.

In another embodiment, powder compositions consisting essentially of a therapeutically effective amount of TXA and suitable excipients that facilitate local administration and methods of use to treat bleeding and wounds. The amount of TXA ranges between 70.1% and 99.5% w/w.

In another embodiment, powder compositions consisting essentially of a therapeutically effective amount of TXA, optionally one or more antibiotics, with suitable excipients that facilitate local administration and methods of use to treat bleeding and wounds.

In another embodiment, powder compositions consisting essentially of a therapeutically effective amount of TXA, optionally one or more anesthetic agent, with suitable excipients that facilitate local administration and methods of use to treat bleeding and wounds.

In another embodiment, powder compositions consisting essentially of a therapeutically effective amount of TXA, optionally one or more antibiotics, optionally one or more anesthetic, and combination of thereof with suitable excipients that facilitate local administration and methods of use to treat bleeding and wounds.

In another embodiment, powder compositions consisting essentially of a therapeutically effective amount of TXA, optionally one or more antibiotics, optionally one or more anesthetic, cyclodexrin and combination of thereof with suitable excipients that facilitate local administration and methods of use to treat bleeding and wounds.

In yet another embodiment, this patent application is to provide powder compositions consisting essentially of a therapeutically effective amount of TXA, optionally one or more antibiotics, optionally one or more anesthetic, and a hydrophilic polymer which can absorb exudates from the wound and form a gel.

In yet another embodiment, the powder compositions containing TXA consists of hyaluronic acid, sodium hyaluronate, chondroitin sulfate and/or collagen. In yet another embodiment, the powder composition of TXA consists of other actives such as aprotinin, epsilon aminocaproic acid (EAA), bromelain or combinations thereof.

In yet another embodiment, particle size of the TXA powder composition should be greater than 10 microns but less than 200 microns to increase nasal deposition and minimize deposition in the lungs when administered as a nasal delivery system. In this case, the “particle size” is defined as D90 which means 90% of the particles are below the particle size value. In other words, for delivery in the nose, the D90 value should be between 10 and 200 microns

In yet another embodiment, particle size of the TXA powder composition should be less than 10 microns to decrease nasal deposition and increase deposition in the lungs when administered as a nasal delivery system.

In yet another embodiment, particle size of the TXA powder composition should be less than 50 microns. This formulation is for topical application and must be smooth to feel.

The powder composition of TXA can be housed in different delivery systems such as a pouch, a tube, a bottle, a bottle with a perforated cap, and a spray can.

In certain embodiments, the therapeutically effective amount of the TXA is between 70.1 and 99.5% w/w.

In certain embodiments, the therapeutically effective amount of the aprotinin is between 0.1 and 29% w/w.

In certain embodiments, the therapeutically effective amount of the epsilon-aminocaproic acid (EACA) is between 0.1 and 29% w/w.

The amount of antibiotic ranges between 0.1 and 29% w/w.

The amount of local anesthetic ranges between 0.1 and 29% w/w.

The amount of hydrophilic polymer ranges between 0.1 and 29% w/w.

The amount of cyclodextrin ranges between 0.1% and 29% w/w.

The amount of hydrophilic polymer which can absorb exudates from the wound and form a gel, ranges between 0.1% and 10% w/w.

### BRIEF DESCRIPTION OF THE DRAWINGS

FIG. 1 shows the cascade of events happening during the wound healing process.

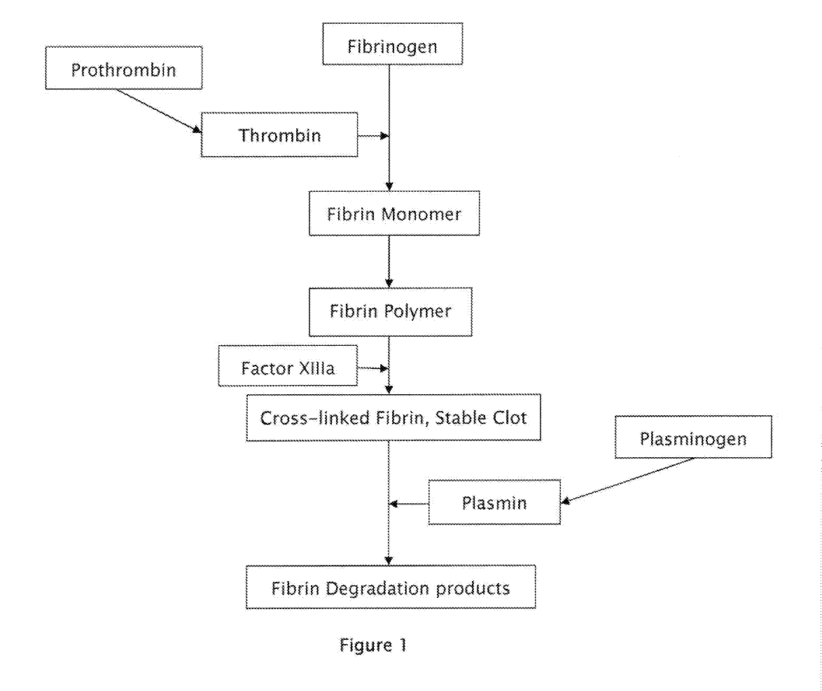

### DETAILED DESCRIPTION

This disclosure relates to powder composition of an antifibrinolytic agent, TXA, for the treatment of bleeding and wounds. In certain embodiments, TXA is administered with one or more antibiotics that are appropriate for internal and external administration. In certain embodiments, the TXA is administered with one or more anesthetic agent that are appropriate for internal and external administration.

The current patent application discloses the composition and application of TXA for the healing of wound by controlling the bleeding. This application refers only to the powder formulations of TXA optionally with other actives along with suitable excipients.

In certain embodiments, the antifibrinolytic is administered with one or more antibiotics.

In certain embodiments, the antifibrinolytic is administered with one or more anesthetic agents.

In certain embodiments, the antifibrinolytic is administered with one or more hydrophilic polymer, which can absorb exudates from wound and swell.

In certain embodiments, the antifibrinolytic is administered with hydrophilic substances such as chondroitin sulfate, collagen, sodium carboxymethyl cellulose, which can absorb exudates from wound and swell.

The powder composition of TXA in this patent application can be applied to mucosal or non-mucosal surfaces. But it is not for an oral administration.

### Definition of Terms Used

Unless defined otherwise, all technical and scientific terms used herein have the same meanings as commonly understood by one of ordinary skill in the art. In describing and claiming the present invention, the following terminology will be used in accordance with the definitions set out below.

As used herein and in the claims, the singular forms “a”, “an”, and “the” include plural reference unless the context clearly dictates otherwise. For example, reference to “an excipient” is a reference to one or more excipients and equivalents thereof known to those skilled in the art. Reference to “an antibiotic” includes a mixture of two or more antibiotics known to those skilled in the art,

The term “about” is used to indicate that a value includes the standard level of error for the substance or method being employed to determine the value. The terms “comprise,” “have” and “include” are open-ended linking verbs. Any forms or tenses of one or more of these verbs, such as “comprises,” “comprising,” “has,” “having,” “includes” and “including,” are also open-ended.

The “effective” amount of a drug or pharmacologically active agent is a sufficient amount to provide the desired pharmacological effect, e.g., reduction of bleeding.

The terms “local administration” and “locally administering” as used herein refer to treatment of bleeding by administering at sites proximate to the wound or where it is bleeding.

“Excipients” are compounds used in the formulation along with the active ingredients, I.e., TXA, antibiotics or local anesthetics. The drug(s) have to be stable in the dosage form along with the excipients throughout the shelf-life of the pharmaceutical dosage form.

“Pharmaceutically Acceptable Materials” refers to those compounds or materials, which are suitable for use in contact with tissues or organs of humans and animals without excessive toxicity, irritation, allergic response or any other problems.

“Chemical stability” with respect to the active agent means that an acceptable percentage of degradation products are produced by chemical pathways such as hydrolysis, thermal degradation or oxidation during the shelf-life of the product.

“Physical stability” with respect to the active agent means that an acceptable percentage of aggregates, loss of original color or discoloration, crystals, visible mold/fungus is formed during storage of the perfume.

Bleeding is the term commonly used to describe blood loss. It can refer to blood loss inside the body (internal bleeding) or blood loss outside of the body (external bleeding). Typically, internal bleeding occurs when blood leaks out through damage to a blood vessel or organ. External bleeding occurs either when blood exits through a break in the skin due to a cut or wound. Traumatic wound is caused by an injury. Many medical conditions such as liver disease, lung cancer, etc. can cause bleeding.

The main uses of antibiotics are used for the treatment or prevention of bacterial or microbial infections. Antibiotics are administered for the treatment of infection as part of an empirical therapy or a definitive therapy. In the empirical therapy, a patient has proven or suspected infection, but the responsible microorganism is not yet unidentified.

Once the microorganism is identified definitive antibiotic therapy can commence.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage.

The term “anesthetic” refers to a substance that that causes loss of sensation and therefore induces insensitivity or low sensitivity to pain.

The term a “non-oral topical composition” means that the composition is not to be administered orally.

Burn is an injury caused by exposure to heat or flame. Burns have a variety of causes, including: scalding from hot, boiling liquids; chemical burns; electrical burns; fires, including flames from matches, candles, and lighters; excessive sun exposure. Burns are divided into three categories—first degree, second degree, third degree and fourth degree. In first-degree burn, the skin becomes red and non-blistered. The second-degree burn is depicted by blisters and thickening of skin. Some blisters can pop open producing inflammation and wetness. There is a widespread thickness and white, leathery appearance in the third-degree burn. Third-degree burns carry the most risk for complications, such as infections, blood loss, and shock. Sepsis, or a bloodstream infection, can occur in the most severe cases. In fourth-degree burns, the damage of third-degree burns extends beyond the skin into tendons and bones.

Method of Treatment

In one aspect, provided herein is a method for treating bleeding in a subject in need thereof, which comprises administering to the subject a pharmaceutical powder composition comprising TXA. The composition comprises excipients that facilitates drug administration. The final powder formulation must show satisfactory physical and chemical stability throughout the shelf-life of the product. In another embodiment, the composition consists essentially of or consists of a therapeutically effective amount of TXA, one or more antibiotics, and suitable excipients. In yet another embodiment, the composition consists essentially of or consists of a therapeutically effective amount of TXA, one or more anesthetic agent, and suitable excipients. In another embodiment, the composition consists essentially of or consists of a therapeutically effective amount of TXA, optionally one or more antibiotics, optionally one or more anesthetic, and suitable excipients. In another embodiment, the composition consists essentially of or consists of a therapeutically effective amount of TXA, optionally one or more anti-inflammatory agent and suitable excipients. In another embodiment, the composition consists essentially of or consists of a therapeutically effective amount of TXA, optionally one or more antibiotics, optionally one or more anesthetic, optionally one or more anti-inflammatory agent, and suitable excipients.

The antibiotics to be in the powder TXA composition can be, e.g., penicillins, cephalosporins, glycopeptides, amoniglycosides, tetracyclins, fluoroquinolones, and quinolones. The specific antibiotics can be moxifloxacin, mupirocin, erythromycin, sulfacetamide, sulfadiazine, mafenide, tetracycline, bacitracin, neomycin, vancomycin, teicoplanin, amikacin, tobramycin, streptomycin, doxycycline, clarithromycin, clindamycin, Lincomycin, ciprofloxacin, or polymyxin B. One or more antibiotics can be selected from the list cited in this paragraph.

The anesthetics can be, e.g., lidocaine, prilocaine, tetracaine and combinations thereof.

The anti-inflammatory agent(s) are selected from the group consisting of bromfenac, diclofenac, ketoprofen, ketorolac, ibuprofen, naproxen, fenoprofen, etodolac, indomethacin and combinations thereof.

The compositions described herein are administered at the site of a wound or cut of the skin or mucosa from which blood had flown or it is flowing.

Powder TXA composition to treat skin wounds.

Powder TXA composition to be used during surgery.

Powder TXA composition to treat nasal bleeding.

Powder TXA composition to be applied in body cavities.

In certain embodiments, the composition comprises of an excipient or carrier that permits spraying of the composition, and the composition is administered by local spraying, such as nasal spray.

In certain embodiments, the composition comprises of an excipient or carrier that permits spraying of the composition, and the composition is administered locally during surgical procedures.

For nasal spray and surgical procedure, the excipient or carrier can be sterile. The composition can further comprise of any drugs for treating diseases of the nose.

Aprotinin is the small protein bovine pancreatic trypsin inhibitor (BPTI), an antifibrinolytic molecule that inhibits trypsin and related proteolytic enzymes. Aprotinin was used as a medication administered by injection to reduce bleeding during complex surgery, such as heart and liver surgery. Its main effect is the slowing down of fibrinolysis, the process that leads to the breakdown of blood clots.

Epsilon-aminocaproic acid is a derivative and analogue of the amino acid lysine, which makes it an effective inhibitor for enzymes that binds reversibly to the Kringle domain of plasminogen and blocks the binding of plasminogen to fibrin and its activation to plasmin. With NO activation of plasmin, there is a reduction in fibrinolysis. This consequently reduces the amount of bleeding post-surgery. Epsilon-aminocaproic acid is FDA-approved for use in the treatment of acute bleeding due to elevated fibrinolytic activity.

Collagen, the protein that gives the skin its tensile strength, plays a key role in each phase of wound healing. It attracts cells, such as fibroblasts and keratinocytes, to the wound, which encourages debridement, anglogenesis, and reepithelialization. In addition, collagen provides a natural scaffold or substrate for new tissue growth.

Collagen dressings stimulate new tissue growth and encourage the deposition and organization of newly formed collagen fibers and granulation tissue in the wound bed. These dressings chemically bind to matrix metalloproteinases (MMPs) found in the extracellular fluid of wounds. MMPs normally attack and break down collagen, so it's thought that wound dressings containing collagen give MMPs an alternative collagen source, leaving the body's natural collagen available for normal wound healing.

Bromelain is used for reducing swelling (inflammation), especially of the nose and sinuses, after surgery or injury. It is also used for hay fever, treating a bowel condition that includes swelling and ulcers (ulcerative colitis), removing dead and damaged tissue after a burn (debridement), preventing the collection of water in the lung (pulmonary edema), relaxing muscles, stimulating muscle contractions, slowing clotting, improving the absorption of antibiotics, preventing cancer, shortening labor, and helping the body get rid of fat.

Hyaluronic acid is a non-sulphated glycosaminoglycan and is composed of repeating polymeric disaccharides of D-glucuronic acid.

Hyaluronic acid regulates several aspects of tissue repair, including activation of inflammatory cells to enhance immune response during tissue injury and wound healing.

Sodium hyaluronate is the sodium salt of hyaluronic acid, a glycosaminoglycan found in various connective, epithelial, and neural tissues. Sodium hyaluronate used as a surgical aid in variety of surgical procedures such as eye surgery, plastic surgery etc. Sodium hyaluronate is also used to coat the bladder lining in treating interstitial cystitis.

Chondroitin sulfate is a sulfated glycosaminoglycan composed of a chain of alternating sugars (N-acetylgalactosamine and glucuronic acid). It is usually found attached to proteins as part of a proteoglycan. Chondroitin sulfate is an important structural component of cartilage and provides much of its resistance to compression. Formulated with collagen and wound dressing matrix, one product that uses chondroitin sulfate is the veterinary wound gel Chondroprotec®, which is applied over scrapes, burns, and lesions and serves to keep the wound moist and promote healing.

Cyclodextrin: Cyclodextrins (CD) are a family of compounds made up of sugar molecules bound together in a ring (cyclic oligosaccharides). Exposure of starch to an enzyme called cyclomaltodextrin glucanotransferase, naturally excreted by B. macerans, yields a mixture of six-, seven- and eight-member rings corresponding to α-CD, β-CD and γ-CD, respectively. Cyclodextrins form host-guest complexes with hydrophobic molecules. Cyclodextrins can solubilize hydrophobic drugs in pharmaceutical applications, and crosslink to form polymers used for drug delivery. For this patent application, the cyclodextrin can be one or more the following—α-cyclodextrin (6 membered sugar ring molecule), β-cyclodextrin (7-membered sugar ring molecule), γ-cyclodextrin (8-membered sugar ring molecule), hydroxypropyl betacyclodextrin, sulfobutylether betacyclodextrin, and randomly methylated beta cyclodextrin.

The pharmaceutical composition can further comprise a preservative. Non-limiting examples of the preservative are benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, meta-cresol, methylparaben, nitromersol, o-phenyl phenol, parabens, phenol, phenylmercuric acetate/nitrate, propylparaben, sodium benzoate, sorbic acids and salts, β-Phenylethyl alcohol, thimerosal.

Powder Formulations

During the development of any powder formulation, the following properties are considered and evaluated—Flow, surface charge, granulation, mass-volume relationship (bulk density), wetting properties, particle size, hygroscopicity etc. Powders can be either free flowing or non-free flowing. The flow properties are affected by particle size, density, shape, electrostatic charge, and adsorbed moisture. Adsorption of moisture may convert a free-flowing powder to non-free flowing during storage (The Theory and Practice of Industrial Pharmacy, by L. Lachman, H. A. Lieberman and J. L. Kanig, 3^{rd} edition, 1986).

Powders for external use should have particle size not more than 150 microns to avoid any sensation of grittiness, which could irritate traumatized skin (Remington, The Science and Practice of Pharmacy, 20^{th} Edition, A. R. Gennaro, 2000). In this patent, we are proposing to keep the particle size of the TXA powder formulation for topical use below 25 microns. Dusting powders usually contain starch, talc and zinc stearate. Absorbable dusting powder, USP contains starch treated with epichlorohydrin. Dusting powders are available in sifter-top containers or pressure aerosols.

Pressure Aerosol powders have the advantage of protection from air/oxidation, moisture, contamination and convenience of application.

Insufflations are finely divided powders introduced in body cavities such as ears, nose, throat and vagina using an insufflator (powder blower).

Trituration is the method of dilution of powders. Normally 1 part of drug is diluted with 1 part of diluent with thorough mixing. The process is continued till all the diluent is added.

According to USP/NF, pharmaceutical aerosols are packaged under pressure and contain therapeutically active ingredients that are released upon activation of an appropriate valve system. Powder formulation can be suspended in a propellant or a mixture of propellants. Chloro or hydrofluoro carbons are some of the commonly used propellants. Problems associated with pharmaceutical aerosols are—agglomeration, caking, particle-size growth, and valve clogging. Lubricants such as isopropyl myristate, or dispersing agents such as lecithin are used to solve some of the issues. The moisture content should be kept low between 100 to 300 ppm.

Following are the key advantages of this invention:
1. The formulation is safe, and easy to use
2. Small amount of formulation is needed to apply to affected area.
3. The formulation is applied to the site of action thereby reducing the side-effects in other parts of body.

With the following examples, one skilled in the art, can understand and use the present invention.

### Pharmaceutical Compositions

### Example 1

The powder Formulation 1 contains TXA as active ingredient with combination of antibiotics.

TABLE I
Composition of a TXA Formulation # 1

| Ingredient | Percent w/w |
|---|---|
| Tranexamic acid | 70.5 |
| Hyaluronic acid | 8 |
| Neomycin | 10 |
| Bacitracin | 9 |
| Sulfacetamide | 2.5 |

### Example 2

The powder Formulation 2 contains TXA as active ingredient with local anesthetic.

TABLE II
Composition of a TXA Formulation # 2

| Ingredient | Percent by weight |
|---|---|
| Tranexamic acid | 75 |
| Maltodextrin | 17 |
| Hyaluronic acid | 5 |
| Prilocaine | 3 |

### Example 3

The powder Formulation 3 contains TXA and Aprotinin as active ingredients with antibiotic and polymer.

TABLE III
Composition of a TXA Formulation # 3

| Ingredient | Percent by weight |
|---|---|
| Tranexamic acid | 72 |
| Aprotinin | 13 |
| Vancomycin | 5 |
| Hydrophilic Polymer | 6 |
| Hyaluronic acid | 4 |

### Example 4

The powder Formulation 4 contains TXA as active ingredient with combination of antibiotic and local anesthetic to treat wound. The product also contains turmeric powder, which is used as a wound healing agent in the Ayurvedic medicines. It also contains a buffer, pH 7.4.

TABLE IV
Composition of TXA Formulation

| Ingredient | Percent by weight |
|---|---|
| Tranexamic acid | 84 |
| Moxifloxacin | 4 |
| Turmeric powder | 3 |
| Lidocaine | 4 |
| Phosphate buffer for pH 7.4 | 5 |

### Example 5

The powder Formulation 5 contains TXA and bromelain as active ingredients with cyclodextrin and sodium hyaluronate.

TABLE V
Composition of a TXA Formulation # 5

| Ingredient | Percent by weight |
|---|---|
| Tranexamic acid | 73 |
| Sodium hyaluronate | 12 |
| Bromelain | 5 |
| Cyclodextrin | 10 |

### Example 6

The powder Formulation 6 contains TXA and Epsilon-aminocaproic acid as active ingredients with cyclodextrin for surgical procedure.

TABLE VI
Composition of a TXA Formulation # 6

| Ingredient | Percent by weight |
|---|---|
| Tranexamic acid | 85 |
| Epsilon-aminocaproic acid | 6 |
| Sodium hyaluronate | 2 |
| Cyclodextrin | 2 |
| Phosphate buffer for pH 7.4 | 5 |

### Example 7

The powder Formulation 7 contains TXA as active ingredient with combination of antibiotics for nasal delivery system. It also contains a local anesthetic—pramoxine.

TABLE VII
Composition of TXA for nasal spray, Formulation #7

| Ingredient | Percent by weight |
|---|---|
| Tranexamic acid | 71.5 |
| Neomycin | 2.5 |
| Bacitracin | 3.5 |
| Polymyxin | 12.5 |
| Pramoxine | 10 |

### Example 8

The powder Formulation 8 contains TXA as active ingredient with antibiotics and hydrophilic polymers to treat oozing wounds.

TABLE VIII
Composition of TXA for oozing wounds, Formulation #8

| Ingredient | Percent by weight |
|---|---|
| Tranexamic acid | 71.5 |
| Neomycin | 2.5 |
| Bacitracin | 3.5 |
| Polymyxin | 10.5 |
| Sodium carboxymethyl cellulose | 4 |
| Sodium hyaluronate | 8 |

In conclusion, various powder compositions containing TXA as the main active ingredient have been presented. The powder formulation is filled in various primary packaging containers such as bottles, pouches, spray cans etc. The powder is sprinkled or sprayed over an internal or external wound. Various other actives promoting wound healing, antibiotics preventing the infections, local anesthetics, anti-inflammatory agents and others such as bromelain, EAA, aprotinin are incorporated into the TXA formulations.

1) (canceled)

2) (canceled)

3) (canceled)

4) (canceled)

5) (canceled)

6) (canceled)

7) (canceled)

8) (canceled)

9) (canceled)

10) (canceled)

11) (canceled)

12) (canceled)

13) (canceled)

14) (canceled)

15) (canceled)

16) (canceled)

17) (canceled)

18) (canceled)

19) (canceled)

20) (canceled)

21) A wound-healing powder composition applied locally to mucosal or non-mucosal surfaces of the wound comprising (a) tranexamic acid in an amount from 70.1 wt % to 99.5% wt %, (b) one or more antibiotic in an amount from 1 wt % to 10 wt %, (c) optionally one or more of aprotinin, bromelain, and epsilon-aminocaproic acid in an amount from 1 wt % to 15 wt %, (d) optionally a local anesthetic in an amount from 0.1% to 5%, (e) optionally an anti-inflammatory agent in an amount from 0.1% to 5%, (f) one or more hydrophilic polymer(s), (g) optionally a cyclodextrin, (h) a preservative, and (I) excipient(s).

22) A wound-healing powder composition of claim 21 wherein one or more antibiotic(s) are selected from the group consisting of penicillins, cephalosporins, glycopeptides, amoniglycosides, tetracyclines, fluoroquinolones, quinolones, moxifloxacin, mupirocin, erythromycin, sulfacetamide, sulfadiazine, mafenide, tetracycline, bacitracin, neomycin, vancomycin, teicoplanin, amikacin, tobramycin, streptomycin, doxycycline, clarithromycin, clindamycin, lincomycin, ciprofloxacin, or polymyxin B and mixtures thereof.

23) A wound-healing powder composition of claim 21 wherein one or more local anesthetic(s) are selected from the group consisting of lidocaine, prilocaine, tetracaine, pramoxine and mixtures thereof.

24) A wound-healing powder composition of claim 21 wherein one or more anti-inflammatory agent(s) are selected from the group consisting of bromfenac, diclofenac, ketoprofen, ketorolac, ibuprofen, naproxen, fenoprofen, etodolac, indomethacin and combinations thereof.

25) A wound-healing powder composition of claim 21 wherein one or more hydrophilic polymer(s) are selected from the group consisting of collagen, sodium hyaluronate, chondroitin sulfate, hyaluronic acid, sodium carboxymethyl cellulose and mixtures thereof.

26) A wound-healing powder composition of claim 21 wherein one or more cyclodextrin(s) are selected from the group consisting of α-cyclodextrin, β-cyclodextrin, γ-cyclodextrin, hydroxypropyl β-cyclodextrin, sulfobutylether β-cyclodextrin, randomly methylated β-cyclodextrin and mixtures thereof.

27) A wound-healing powder composition of claim 21 wherein the preservatives selected from the group consisting of benzalkonium chloride, benzethonium chloride, benzoic acid, sodium benzoate, benzyl alcohol, boric acid, cetylpyridinium chloride, cetyltrimethyl ammonium bromide, chlorobutanol, chlorocresol, chorhexidine gluconate, chlorhexidine acetate, cresol, ethanol, imidazolidinyl urea, metacresol, methylparaben, nitromersol, o-phenyl phenol, parabens, phenol, phenylmercuric acetate, phenylmercuric nitrate, propyl paraben, sorbic acids, potassium sorbate, propionic acid, β-Phenylethyl alcohol, thimerosal and combinations thereof.

28) A wound-healing powder composition of claim 21 wherein the particle size of the powder ranges from 0.1 micron to 200 microns.

29) A method of administration of wound-healing powder composition comprising spraying or sprinkling the powder from a bottle, pouch, can or a spray-bottle over an internal or external wound.

30) A method of administration of wound-healing powder composition in claim 29 comprising tranexamic acid in an amount from 70.1 wt % to 99.5% wt % and excipients in an amount from 0.5 wt % to 29.9 wt %.
